(19) United States
(12) Patent Application Publication
(10) Pub. No.: US 2006/0106005 A1
(43) Pub. Date: May 18, 2006

(54) Funtionalised metallocenes as anticancer drugs

(76) Inventors: Patrick Columba McGowan, Headingley (GB); Richard John Knox, Salisbury (GB)

Correspondence Address: MOORE & VAN ALLEN PLLC, P.O. BOX 13706, Research Triangle Park, NC, 27709, US

(21) Appl. No.: 10/520,477

(22) PCT Filed: Jul. 4, 2003
(86) PCT No.: PCT/GB03/02886

§ 371 Date: Sep. 29, 2005

(51) Int. Cl. A61K 31/555; C07F 17/00

(52) U.S. Cl. 514/184; 546/2

(57) ABSTRACT

The invention provides metallocene compounds of formula 1 for use as medicaments in the treatment of cancer. 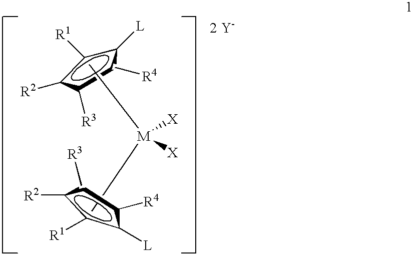
wherein R¹, R², R³ and R⁴ represent a combination of H, alkyl, aryl or trimethylsilyl; L represents side chain substituents, at least one of which contains a group which enables the compound to become water-solubilised; X is halo, alkoxy, acetate or H₂O; Y is a counter-ion; M is a metal; and n=1 or 2. The invention also provides novel compounds of the formula 1 wherein at least one of the groups L comprises a quaternary tetraalkylammonium group. The compounds have been shown to have significantly greater activity in the treatment of cancer than the compounds known from the prior art.

### FIELD OF THE INVENTION

This invention relates to the use of functionalised metallocenes as anti-tumour reagents.

### BACKGROUND OF THE INVENTION

A major problem with chemotherapy is the lack of selectivity of drugs for cancer cells. Consequently, the side effects of such treatment can be very unpleasant, and often worse than the symptoms of the actual disease. One of the best anticancer drugs known to those skilled in the art has been cisplatin, and the action and mechanism of this drug is now fairly well understood. However, the toxic side effects of cisplatin include nausea, vomiting, neuropathy, ototoxicity (tinnitus/hearing loss) and nephroxicity.¹ Although treatment with an alternative platinum based drug (carboplatin) lessens these side effects, this material has greater bone marrow toxicity.² Nevertheless, these compounds do find widespread medical usage in this field and the high specificity of cisplatin in treating testicular cancer suggests that it should be possible to synthesise other metal-based drugs to treat specific tumour types.

There has been a vigorous quest to locate effective new anti-cancer drugs, preferably having fewer side effects. Furthermore, new approaches are needed to tackle the problem of tumour resistance to cisplatin. For example, ovarian cancer patients initially respond well to the drug, but eventually develop resistance and succumb to the disease.² Amongst the candidates for antitumour reagents have been the early transition metal compounds, metallocene dichlorides[(C₅H₅)₂MCl₂, M=Ti, V, Nb, Mo, Re]. The antitumour activity of both titanocene (bis-cyclopentadienyl titanium)dichloride [(C₅H₅)₂TiCl₂] or Cp₂TiCl₂ and vanadocene(bis-cyclopentadienyl vanadium)dichloride [(C₅H₅)₂VCl₂] or Cp₂VCl₂ has been established against various animal and xenografted human tumours.^{3,4}

Titanocene dichloride is one of the most effective anti-tumour agents of this type and is currently undergoing Phase II clinical trials.⁵ On a toxicological and pharmacokinetic level, both Cp₂TiCl₂ and vanadocene dichlorides are interesting and effective antitumour drugs which differ from organic anti-tumour reagents and platinum cytostatic drugs by their pattern of toxicity and their pharmacokinetic behaviour.^{3,6} In vivo research has shown xenografted tumours of the colon, head, breast, rectum, lung and stomach to be significantly sensitive to Cp₂TiCl₂ and generally to a greater extent than is found with cisplatin.⁶

Cp₂VCl₂ has been shown to have different activity than Cp₂TiCl₂; for example, with respect to their activity against different lung carcinomas.⁷ For LX1, which is a histologically unclassified tumour which does not respond to most clinically used cytostatic drugs, Cp₂TiCl₂ displayed no significant anti-tumour activity, whereas Cp₂VCl₂ showed significant activity.⁸ The consequence of this is that the nature of the active species is unknown and the administration of the compound as a drug can be difficult.

By their very nature, Cp₂TiCl₂ and Cp₂VCl₂ are flawed as drugs because of hydrolysis problems.^{6,9} Hence, there is a requirement for the development of compounds which do not suffer from problems associated with aqueous instability, and it is an object of the present invention to provide such materials and to satisfy the requirements for new anti-cancer drugs. The present invention, therefore, seeks to provide a range of stable and active metallocenes as anticancer compounds.

It has been found that the stability, and thereby the solubility, of metallocene dichlorides in aqueous solutions is improved when the compound is ionic. Limited prior art exists in this area; however, of particular note is the ionic compound [(C₅H₅)₂TiCl(NCCH₃)]⁺ (FeCl₄)⁻, which was the subject of earlier studies relating to antitumour drugs.³ It has also been established that ionic titanocenes of this type have been more effective against head and neck xenografts than both neutral titanocene and vanadocene dichlorides.¹⁰

The present inventors, in the co-pending patent application published as WO 01/42260 have disclosed methods for the synthesis of metallocene halide salts having at least one cyclopentadiene group substituted by a basic group. However, that application contains no disclosure of the use of these materials for the treatment of cancer and, consequently, no data are provided for the success or otherwise of these materials in such treatments.

Most particularly, the present invention is concerned with various titanocene, vanadocene and molybdocene dichlorides, their synthesis and characterisation, and their use in the treatment of diseases, primarily cancer. The invention also involves an investigation of the efficiency of such compounds.

Thus, there are provided a number of water soluble metallocene halide salts, which have the potential to act as potent and effective anticancer agents. In addition, evidence is presented with regard to the increased stability and enhanced activity of these ionic metallocenes with respect to different sets of cancer cell lines.

### STATEMENTS OF INVENTION

According to the present invention, there is provided a metallocene compound 1 for use as a medicament in the treatment of cancer.

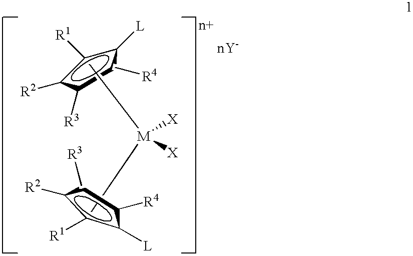

In 1: R¹, R², R³ and R⁴ represent a combination of H, alkyl, aryl or trimethylsilyl;
L represents side chain substituents, at least one of which contains a group which enables the compound to become water-solubilised;
X is halo, alkoxy, acetate or H₂O;
Y is a counter-ion;
M is a metal; and
n=1 or 2.

Specifically, the value of n is determined by the requirement for the molecule to show electrical neutrality and, consequently, is determined by the number of substituents L which contain a group which enables the compound to become water-solubilised. Thus, when the molecule contains one substituent L which enables the compound to become water-solubilised, n=1 and, when the molecule contains two such groups, n=2.

Preferably the metal is titanium, vanadium, niobium or molybdenum. Typical counter-ions include halide, acetate, tetrafluoroborate or hexafluorophosphate ions. The preferred titanocene, vanadocene niobiocene and molybdocene compounds are most preferably in the form of the dichloride salts. The compounds may be in the form of solvates or pro-drugs.

At least one cyclopentadienyl ring is functionalised by means of the group L such that the compound is water soluble. Preferably the at least one cyclopentadienyl ring is functionalised by a group L that carries a pendant Lewis base which confers aqueous solubility, such as an amino-functionalised side chain which can be quaternised.

Typically, in order to funtionalise the cyclopentadienyl ring, the group L comprises an alkyl group with a terminal Lewis base and preferably L has the formula
—(CH₂)ₙZ
wherein n is an integer from 1 to 20 and Z comprises an amino group, for instance a secondary amino group, a particularly favoured example being a —(CH₂)₂N(CH₂)₅ group, which may be quaternised to provide compounds such as those of formula 2 or 3. These compounds may comprise trialkyl ammonium halides, such as the compound of formula 2 or, most advantageously, novel tetraalkylammonium compounds including the compound of formula 3.

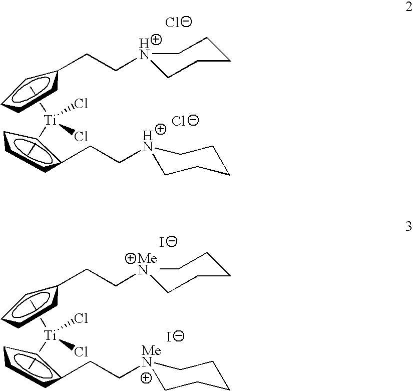

According to the invention, at least one of the L groups comprises a functionalised substituent capable of enabling the compound to become water-solubilised, and both groups may comprise such substituents. However, on the occasions when only one of the L groups comprises such a functionalised substituent,

then the L group on the other cyclopentadienyl ring may comprise any substituent not associated with conferring aqueous solubility on the molecule, typical examples being alkyl, aryl, aralcyl or, preferably, trialkylsilyl groups, for example, trimethylsilyl groups; alternatively, in such cases, L may be hydrogen.

Particular examples of compounds wherein only one of the L groups comprises a functionalised substituent include those of formulae 4, 5, 6 and 7.

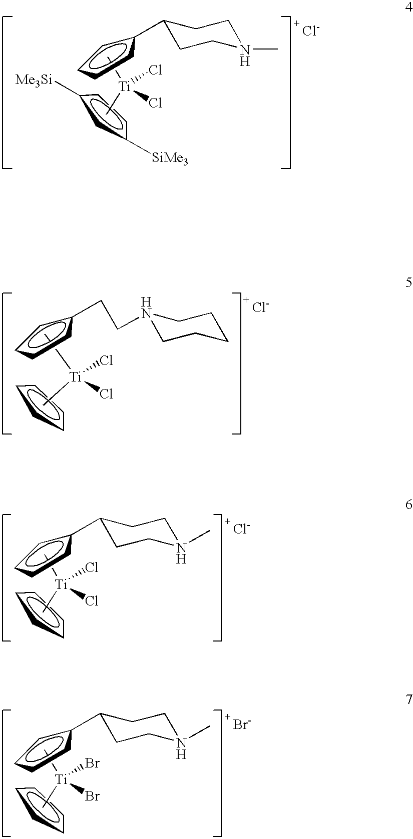

Specifically, the present invention relates to a series of compounds having an ionic feature which is contained within the ligand. This ionic character enables the compounds to overcome the problems of poor water solubility and instability to hydrolysis which are associated with the compounds of the prior art. Thus, the compounds are found to act as potent anti-tumour reagents.

The invention provides a method of treating and/or preventing cancer, which encompasses the administration of a therapeutically effective amount of the compounds 1 to the patient.

Admistration of the compounds of invention comprises of a number of routes including orally, parenterally, topically, nasally or via slow releasing microcamers.

Suitable excipients include, saline, sterile water, creams, ointments, solutions, gels, pastes, emulsions, lotions, oils, solid carriers and aerosols.

The specific amount of compound required will depend on a number of factors, such as the biological activity of the compound used and the age, body and sex of the subject. The subject may be a human or mammalian animal.

The compounds and compositions of the invention can be administered alone or in combination with other compounds. The other compounds may have a biological activity, which complements the activity of the compounds of the invention, e.g., by enhancing its effect in killing tumours or by reducing side effects associated with the compounds of the invention.

### DETAILED DESCRIPTION OF THE INVENTION

Preferably the metal is a group IV metal, more preferably, titanium, an example being [Cp(CH₂)₂NH(CH₂)₅]₂TiCl₂.2HCl 2, which was tested against a number of cell lines, as detailed in Tables 1 and 2.¹¹ Of particular significance is that 2 is much more active when compared to Cp₂TiCl₂; 2 is almost a factor of 10 times more potent than Cp₂TiCl₂.⁵ Indeed, the compounds of the present invention, which show much greater stability in aqueous media than the compounds of the prior art, are generally found to be around 10 times more potent towards certain cancer cell lines than is the case with cisplatin.

In order to determine whether these types of complexes overcome platinum resistance in vitro, the anti-proliferative effects of 2 on cisplatin-resistant A2870 were examined (see Table 3). As ovarian tumours often develop resistance to platinum compounds, these lines present a good model for screening. After 144 hours, cisplatin appears to be 3 times more resistant than 2. The dramatic decrease in resistance factor suggests that these functionalised titanocene dichloride compounds confer a potent anti-proliferative effect on platinum-resistant ovarian tumour cell lines.

Furthermore, changing the group attached to the amino functionality has a dramatic effect on the efficacy of the drugs for A2780 cell line, but not for the cisplatin resistant cell line of A2870-cis, as indicated in Table 4, which includes comparative data for the compounds of formulae 2 and 3. A particular advantage of the compounds of the present invention is their enhanced activity towards cisplatin-resistant cell lines; it is found that the activity of these compounds remains constant whilst the effects of cisplatin diminish.

TABLE 1
The sensitivity of cell lines to Cisplatin and Ti Compounds

| | | | DIFFERENTIAL |
|---|---|---|---|
| CELL LINE | DRUG | IC50 (μM) | (μM) |
| MCF-7 | Cisplatin | 9 | — |
| | 2 | 62 | 6.9 |
| LoVo | Cisplatin | 0.37 | — |
| | 2 | 62 | 167.6 |
| LS 174T | Cisplatin | 0.50 | — |
| | 2 | 36 | 72 |
| A2780 | Cisplatin | 0.82 | — |
| | 2 | 31 | 37.8 |
| A2780-cis | Cisplatin | 2.1 | — |
| | 2 | 28 | 13.3 |
| A2780 | Cisplatin | 1 | — |
| (24 hour) | | | |
| | 2 | 168 | 168 |
| A2780-cis | Cisplatin | 5.4 | — |
| (24 hour) | | | |
| | 2 | 154 | 28.5 |

TABLE 2
The relative sensitivity of cell lines to Cisplatin and Ti Compounds

| CELL LINE | CISPLATIN | 2 |
|---|---|---|
| LoVo | 1 | 1 |
| MCF-7 | 24.3 | 1 |
| LS-174T | 1.35 | 0.58 |
| A2780 | 2.2 | 0.5 |
| A2780cis | 5.7 | 0.45 |

TABLE 3
The relative sensitivity of A2780 and A2780cis cell
lines to Cisplatin and Ti Compounds

| CELL | EXPOSURE | DRUG | |
|---|---|---|---|
| LINE | TIME (hr) | CISPLATIN | 2 |
| A2780 | 144 | 1 | 1 |
| A2780cis | 144 | 2.56 | 0.90 |
| A2780 | 24 | 1.22 | 5.42 |
| A2780cis | 24 | 6.59 | 4.97 |

TABLE 4
The relative sensitivity of A2780 and A278Ocis cell
lines to Ti-2 and Ti-4 Compounds

| CELL LINE | DRUG | IC50(μM)(7 days) |
|---|---|---|
| A2780 | 2 | 99.1 |
| | 3 | 765.6 |
| A2780cis | 2 | 4.3 |
| | 3 | 5.2 |

### REFERENCES

(1) Trimmer, E. E.; Essigmann, J. M. Essays in Biochemistry 1999, 34, 191-211.

(2) Kelland, K. R. problems with carboplatin; Chapman and Hall: London, 1994.

(3) Köpf-Maier, P. Metal Compounds in Cancer Therapy, Chapter 6-Organometallic titanium, vanadium, niobium, molybdenum and rhenium complexes—early transition metal antitumour drugs; Chapman and Hall: London, 1994.

(4) Köpf-Maier, P. Complexes in Cancer Chemotherapy; VCH: Weinheim, New York, 1993.

(5) Christodoulou, C. V.; Eliopoulos, A. G.; Young, L. S.; Hodgkins, L.; Ferry, D. R.; Kerr, D. J. Brit. J Cancer 1998, 77, 2088-2097.

(6) Harding, M. M.; Moksdi, G. J. Current Medicinal Chemistry 2000, 7, 1289-1303.

(7) Murthy, M. S.; Rao, L. N.; Kuo, L. Y. Inorg. Chim. Acta 1988, 152, 1577.

(8) Vendetti, J. Semin. Oncol. 1981, 8, 349.

(9) Toney, J. H.; Marks, T. J. J. Am. Chem. Soc. 1985, 107, 947-953.

(10) Köpf-Maier, P.; Köpf, H. Z. Naturforsch. B 1979, 34, 805.

(11) Allen, O. R.; Knox, R.; McGowan, P. C. Unpublished Results.

1. A method of treating cancer, preventing cancer, or both in a patient, said method comprising administering to the patient a therapeutically effective amount of a metallocene compound of formula 1 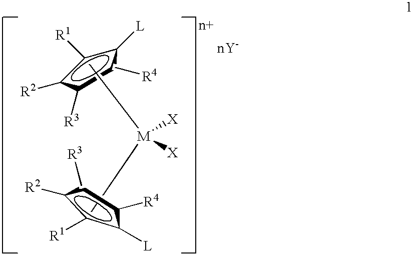
wherein R¹, R², R³ and R⁴ represent a combination of H, alkyl, aryl or trimethylsilyl;
L represents side chain substituents, at least one of which contains a group which enables the compound to become water-solubilised;
X is halo, alkoxy, acetate or H₂O;
Y is a counter-ion;
M is a metal; and
n=1 or 2,
wherein n=1 when one of the side chain substituents L contains a group which enables the compound to become water-solubilised, and n=2 when both of the side chain substituents L contain groups which enable the compound to become water-solubilised, and achieving cancer treatment, cancer prevention, or both in the patient.

2. The method of claim 1 wherein the metal M is titanium, vanadium, niobium or molybdenum.

3. The method of claim 1 wherein the counter-ion Y is a halide, acetate, tetrafluoroborate or hexafluorophosphate ion.

4. The method of claim 1, wherein the metallocene compound is in the form of the dichloride salt.

5. The method of claim 1 wherein the metallocene compound is in the form of a solvate or a pro-drug.

6. The method of claim 1 wherein both groups L are functionalised to enable the compound to become water-solubilised.

7. The method of claim 1 wherein only one group L is functionalised to enable the compound to become water-solubilised.

8. The method of claim 1 wherein L comprises a group which carries a pendent Lewis base.

9. The method of claim 8 wherein the Lewis base is provided by an amino group.

10. The method of claim 9 wherein the amino group is a secondary amino group.

11. The method of claim 10 wherein the secondary amino group comprises a —(CH₂)₂N(CH₂)₅ group.

12. The method of claim 9 wherein the group L has the formula
—(CH₂)_{q}Z
wherein q is an integer from 1 to 20 and Z comprises an amino group.

13. The method of claim 1 wherein the metallocene compound has the formula 2:

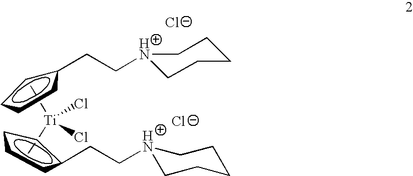

14. The method of claim 1 wherein the metallocene compound has the formula 3:

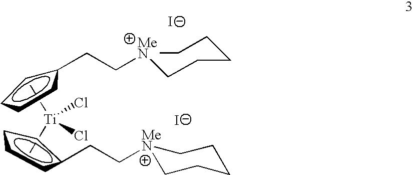

15. The method of claim 1 wherein the metallocene compound has the formula 4:

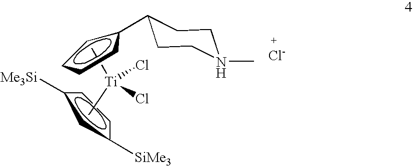

16. The method of claim 1 wherein the metallocene compound has the formula 5:

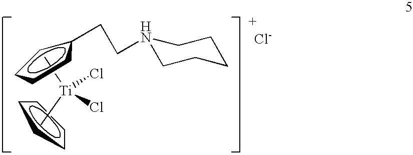

17. The method of claim 1 wherein the metallocene compound has the formula 6:

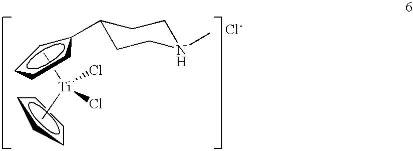

18. The method of claim 1 wherein the metallocene compound has the formula 7:

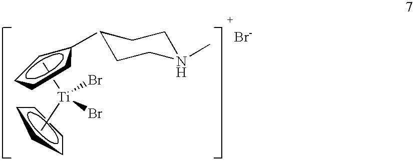

19. The method of claim 1 wherein both groups L comprise a quaternary tetraalkylammonium group.

20. The method of claim 1 wherein only one group L comprises a quaternary tetraalkylammonium group.

21. The method of claim 1 wherein administering to a patient comprises one or more of orally, parenterally, topically, nasally or via slow releasing microcarriers.

22. The method of claim 1 further including one or more excipients comprising saline, sterile water, creams, ointments, solutions, gels, pastes, emulsions, lotions, oils, solid carriers or aerosols.

23. The method of claim 1 wherein the metallocene compound is alone or in combination with at least one other compound.

24. The method of claim 23 wherein said at least one other compound has biological activity.
